(19) United States
(12) Patent Application Publication
(10) Pub. No.: US 2021/0261992 A1
(43) Pub. Date: Aug. 26, 2021

(54) ENZYMES, METHODS, AND HOST CELLS FOR PRODUCING CARMINIC ACID

(71) Applicant: Manus Bio, Inc., Cambridge, MA (US)

(72) Inventors: Ryan Nicholas PHILIPPE, Cambridge, MA (US); Ajikumar Parayil KUMARAN, Cambridge, MA (US); Christine Nicole S. SANTOS, Cambridge, MA (US)

(21) Appl. No.: 17/251,922

(22) PCT Filed: Jun. 12, 2019
(86) PCT No.: PCT/US19/36674

§ 371 Date: Dec. 14, 2020

Related U.S. Application Data
(60) Provisional application No. 62/684,440, filed on Jun. 13, 2018.

(51) Int. Cl. C12P 17/06; C12N 15/52

(52) U.S. Cl. CPC C12P 17/06; C12R 2001/645; C12N 15/52

(57) ABSTRACT

The present invention is related to enzymatic pathways for production of carminic acid, host cells capable of production of carminic acid, and methods for the production of carminic acid and related compounds.

### CROSS REFERENCE TO RELATED APPLICATIONS

This application claims priority to U.S. Provisional Application No. 62/684,440, filed on Jun. 13, 2018, the content of which is hereby incorporated by reference in its entirety.

### SEQUENCE LISTING

This application contains a Sequence Listing, which has been submitted in ASCII format via EFS-Web and is hereby incorporated by reference in its entirety. Said ASCII copy, created on Jun. 10, 2019, is named MAN-017PR_ST25 and is 85 kilobytes in size.

### BACKGROUND

The natural pigment carmine is one of the most frequently used colorants of food, beverages, medicine, cosmetics, and textiles. It is the aluminum salt of carminic acid (CA), a glucosylated anthraquinone. Depending on the pH, the colorant may be in a spectrum from orange to red to purple and is generally known as cochineal or cochineal color.

Carminic acid is extracted from insects, most commonly from the female insect bodies of cochineal (Dactylopius coccus). The insects live on various species of cactus plants, which are cultivated in the desert areas of Mexico, Central and South America, and the Canary Islands. Current industrial production of carmine involves the harvesting of CA from cochineal insects grown on Opuntia ficus-indica cactus plants in commercial plantations. This source is relatively expensive and subject to undesirable quality variation and price fluctuation.

The CA is extracted from the bodies of dried insects with water or alcohol. This approach to extraction results in some amount of insect protein contaminating the colorant product, creating a risk for allergy-related problems. This has prompted the exploration of synthetic chemistry approaches to the production of carmine, although the expense of these processes prohibits their broad application.

Accordingly, a consistent, economical, and scalable process for the production of CA and related compounds is desired.

### SUMMARY OF THE INVENTION

In one aspect, the present invention is related to a host cell for producing carminic acid where the host cell expresses an enzymatic pathway for biosynthesis of carminic acid from polyketide building blocks.

In another aspect, the present invention is related to a method of producing carminic acid where the method includes a step of culturing the microbial cell according to the first aspect of this invention under suitable conditions for producing carminic acid.

Other aspects and embodiments of the invention will be apparent from the following detailed description of the invention.

### BRIEF DESCRIPTION OF DRAWINGS

FIG. 1A shows the chemical structure of carminic acid. FIG. 1B shows the chemical structure of carmine, the aluminum salt of carminic acid.

FIG. 2 shows a biosynthetic pathway for the production of CA.

### DESCRIPTION OF THE INVENTION

In various aspects and embodiments, the invention provides enzymatic pathways, recombinant host cells, and methods for the production of carminic acid (CA) and related compounds.

The biosynthetic source of CA has been the subject of scientific study for some time. While fungi, plants, and bacteria are known to produce a large variety of polyketides, the production of these compounds in insects is very rare. Some species of herbivorous insects of the Aphidoidea (aphids, lice) and Coccoidea (scale insects or mealybugs) families can produce polyketides, though the biosynthetic route(s) by which they do so have not been described.

In various embodiments, the invention provides methods of producing CA or related compounds via microbial fermentation. In various embodiments, the enzymatic pathway for production of CA is expressed in microbial host cells, such as a yeast or bacteria. In some embodiments, the microbial host is a yeast, such as a species of Saccharomyces, Pichia, or Yarrowia, including Saccharomyces cerevisiae, Pichia pastoris, and Yarrowia lipolytica. In some embodiments, the microbial host cell is Yarrowia lipolytica. In some embodiments, the microbial host cell is a bacterium selected from Escherichia spp., Bacillus spp., Corynebacterium spp., Rhodobacter spp., Zymomonas spp., Vibrio spp., and Pseudomonas spp. For example, the bacterial strain is a species selected from Escherichia coli, Bacillus subtilis, Corynebacterium glutamicum, Rhodobacter capsulatus, Rhodobacter sphaeroides, Zymomonas mobilis, Vibrio natriegens, or Pseudomonas putida. In some embodiments, the microbial host cell is E. coli.

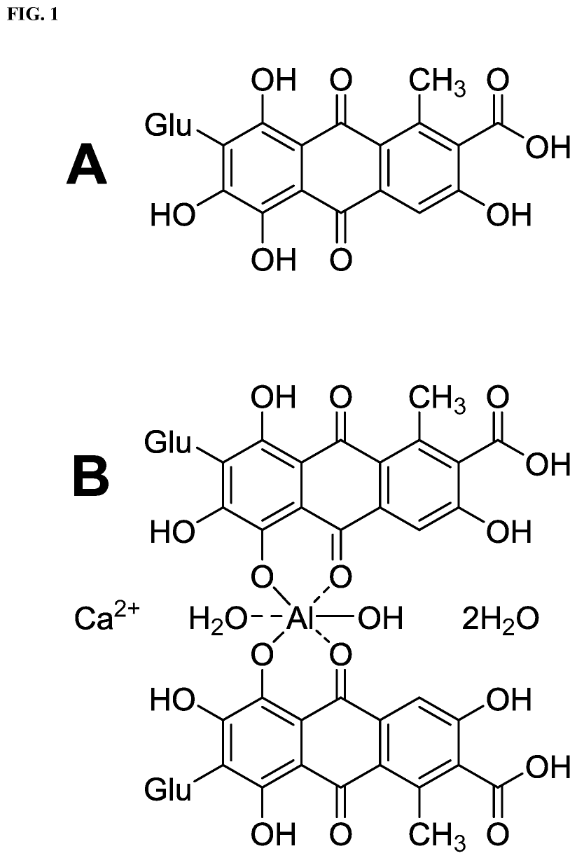

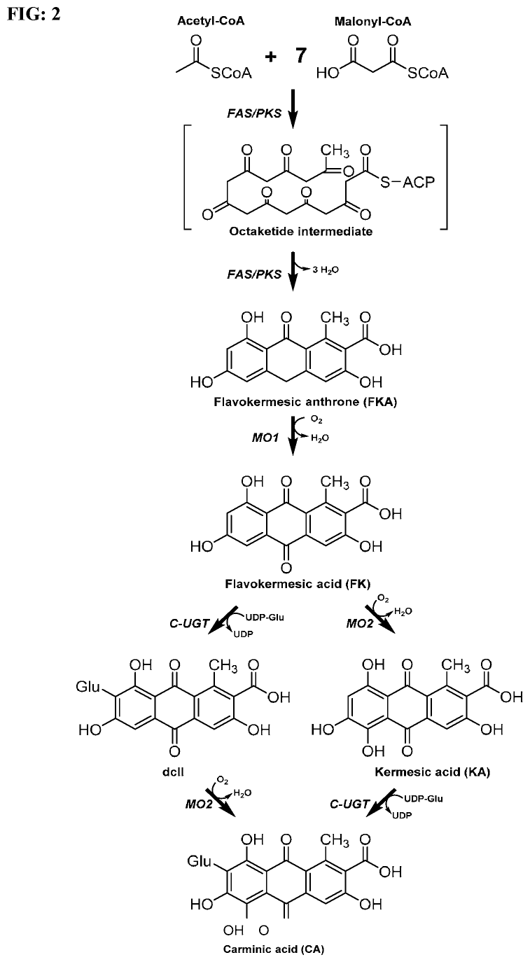

The structure of CA is shown in FIG. 1A. CA is a glucosylated anthraquinone likely derived from polyketide biosynthesis. Carmine, the aluminum salt, is shown in FIG. 1B. Based on precursors to CA identified in a variety of aphid and scale insect species, a proposed biosynthetic pathway is shown in FIG. 2. An enzyme, likely related to a fatty acid synthase (FAS), is believed to be responsible for production of the octaketide that leads to flavokermisic acid anthrone (FKA). FKA is converted either spontaneously or by action of a monooxygenase (MO1) to flavokermesic acid (FK). FK is then converted to either kermesic acid (KA) by the same or a different monooxygenase (MO2), or to FKA 2-C-glucoside (dcII) by a UDP-glycosyltransferase (UGT). These two enzymes then act on the alternate substrates to generate glycosylated CA. Both KA and dcII have been isolated from Dactylopius coccus, indicating that either or both can act as precursors to CA.

In various embodiments, the microbial host cell expresses: (1) a recombinant fatty acid synthase (FAS)/polyketide synthase (PKS) that converts Acetyl-CoA and/or Malonyl-CoA building blocks to flavokermesic anthrone (FKA); (2) a monooxygenase enzyme that converts FKA to flavokermesic acid (FK), and a monooxygenase enzyme that converts FK to kermesic acid (KA), where the monooxygenases can be the same or different; and (3) a C-UGT that glycosylates FK and/or KA substrate. The microbial cell can be cultured to produce CA and/or related compounds by fermentation and can be recovered from host cells and/or culture media.

In exemplary embodiments, one or more enzymes are native enzymes from a bacterial, fungal, plant or insect species, or an engineered variant thereof. There is a genome assembly for Dactylopius coccus publicly available on GenBank (ASM83368v1), as well as Pseudococcus longispinus (PLON). In addition, there are eight different transcriptome assemblies for D. coccus or its endosymbiont Wolbachia sp. in GenBank.

In some embodiments, one or more enzymes are enzymes of Dactyopius coccus, Coccus hesperidum, Porphyrophora polonica, Porphyrophora hamelii, Palmicultor browni, or Pseudococcus longispinus. Multiple insect species produce CA and its precursors FK and dcII (D. coccus, Coccus hesperidum, Porphyrophora polonica, Porphyrophora hamelii). Other species produce only FK and dcII (Palmicultor browni, Pseudococcus longispinus), while many other closely related species produce none of these compounds (e.g., Pseudaulacaspis pentagona). This chemical variation can be exploited to select the particular genes that encode enzymes in the CA biosynthetic pathway. For example, D. coccus will express the FAS/PKS, MO1, MO2, and UGT enzymes, while P. browni will not express MO2 and P. pentagona will not express any of them. Generating a transcriptome of each insect species and comparing the commonalities and differences between the sets of expressed genes will narrow down the list of candidate genes to functionally characterize in order to identify functional enzymes.

In various embodiments, the FAS/PKS enzyme is an insect enzyme or engineered variant thereof. In some embodiments, the FAS/PKS is an enzyme of Dactyopius coccus, Coccus hesperidum, Porphyrophora polonica, Porphyrophora hamelii, Palmicultor browni, or Pseudococcus longispinus; or an engineered variant thereof. An engineered variant can generally comprise from 1 to 50, or from 1 to 20, or from 1 to 10 amino acid modifications independently selected from substitutions, insertions, or deletion. In some embodiments, the engineered variant is at least 50% identical, or at least 75% identical, or at least 90% identical, or at least 95% identical, or at least 98% identical to the parent enzyme.

The enzymes in the insect that possess the polyketide synthase (PKS) and cyclase activities have not been described, and no enzymes in the transcriptome possess similarity to known Type I, Type II, or Type III polyketide synthase. This enzyme is likely evolved from a FAS, since all PKS enzymes are rooted in fatty acid biosynthesis. In some embodiments, the PKS is from an insect known to produce carmine or one of its precursors and can be selected via transcriptome sequencing and functional characterization of candidate genes.

Since enzymes of either Type I, II, or III classes of polyketide synthases (PKS) have not been identified in any insect species, there has been much debate over the origin of compounds such CA. Proposed routes include (1) de novo biosynthesis by some unknown pathway in the insect, (2) biotransformation of a polyketide obtained from the consumed plant, (3) production by an endosymbiotic microbe in the insect, or (4) a pathway combining some or all of the above possibilities. However, while the polyketide pederin is produced in Paederus beetles via endosymbiotic bacteria, there is no evidence of such a source for CA in cochineal they produce CA even when treated with antibiotics to destroy their microbiome. Further, although carmine is produced industrially from cochineal reared on Opuntia ficus-indica cacti, the insects are known to produce CA even when feeding on different plant sources. Moreover, the plants that they feed on have not been demonstrated to produce CA or its precursors. Therefore, all signs point to some unknown endogenous biosynthetic pathway possessed by the insect.

Alternatively, the PKS enzyme is a plant, fungal, or bacterial enzyme that possesses the required octaketide synthase and cyclase activities, which can be selected from a functional screen. In some embodiments, the PKS enzyme is a Type I, Type II, or Type III PKS.

In some embodiments, various modules involved in Type I and Type II polyketide synthases that could be assembled and refactored to create a polyketide synthase system capable of flavokermesic acid anthrone biosynthesis. For example, a functional PKS/cyclase enzyme is assembled from multiple enzymes. Since bacterial and fungal PKS enzymes are formed from multiple modules, an enzyme can be assembled from modules from different enzymes. See WO 2016/198564 or WO 2016/198623, which are hereby incorporated by reference in its entirety. Also see, Andersen-Ranberg, J., et al., Synthesis of C-Glucosylated Octaketide Anthraquinones in Nicotiana benthamiana by Using a Multispecies-Based Biosynthetic Pathway, Chem Bio Chem, 18(19), 1893-1897 (2017).

Polyketides are synthesized by a group of enzymes commonly referred to as polyketide synthases (PKS). Polyketide biosynthesis and PKS are derived from fatty acid biosynthesis and fatty acid synthases (FAS), respectively. However, relative to fatty acid chains, polyketide backbones exhibit great variety with respect to the choice of acyl-CoA building blocks and the degree of reduction of beta-ketone functional groups that result after each round of chain elongation.

All PKS share the ability to catalyze Claisen condensation-based fusion of acyl groups by the formation of C—C bonds with the release of carbon dioxide. This reaction is catalyzed by a beta-KetoSynthase domain (KS). In addition to this domain/active site, synthesis can also depend on, but not exclusively, the action of Acyl Carrier Protein (ACP), Acyl Transferase (AT), Starter Acyl Transferase (SAT), product CYClase (CYC), KetoReductase (KR), DeHydratase (DH), Enoyl Reductase (ER), and C-methyl transferase (Cmet).

The substrates for polyketide synthesis are typically classified into starter and extender units, where the starter unit, including but not limited to acetyl-CoA is the first added unit of the growing polyketide chain. Extender units such as malonyl-CoA, but not exclusively, are then subsequently added to elongate the polyketide chain.

Biosynthetic variability arises from independent control of each round of chain elongation by one module of enzymes within a multimodular PKS (a module refers to a collection of dissociated enzymes). The elongation module consists of enzymes involved in chain extension steps of polyketide biosynthesis, while the initiation module consists of enzymes involved in the non-acetate priming of certain aromatic PKS.

PKS can be categorized as reducing or non-reducing based on the level of modifications found in the final polyketide product. These modifications can either be introduced by the PKS enzyme/active unit, or by post-acting enzymes. Non-reduced polyketides are characterized by the presence of ketone groups (—CH₂—CO—), originating from the starter or extender units either as ketones or in the form of double bonds in aromatic groups. In reduced polyketides a single or all ketones have been reduced to alcohol (CH₂—CHOH—) groups by a KR domain/enzyme, or further to an alkene group (—C═C—) by a DH domain/enzyme, or even further to an alkane group (—CH₂—CH₂—) by an ER domain/enzyme.

At all levels (1° amino acid sequence, 2° protein folds, 3° protein structure, and 4° multi-protein arrangement) the PKS display great diversity, and by these criteria are divided into three types.

Type I PKS systems are typically found in filamentous fungi and bacteria, where they are responsible for the formation of aromatic, polyaromatic, and reduced polyketides. They possess several active sites on the same polypeptide chain and the individual enzyme is able to catalyze the repeated condensation of acyl groups, typically two-carbon unites. The minimal set of domains in Type I PKS includes KS, AT, and ACP. Type I PKS are further subdivided into modular PKS and iterative PKS. Type I iterative PKS are typically found in fungi, while Type I modular PKS are typically found in bacteria. Iterative PKS possess a single copy of each active site type and reuse these repeatedly until the growing polyketide chain has reached a predetermined length. Type I iterative PKS that form aromatic and/or polyaromatic compounds typically rely on PT and CYC domains to direct folding of the formed non-reduced polyketide chain. In contract, Type I modular PKS contain several copies of the same actives sites, organized into repeated sequences of active sites called modules. Each module is responsible for adding and modifying a single ketide unit. Each active site in an individual module is only used once during synthesis of a single polyketide.

Type II PKS systems form aromatic and polyaromatic compounds in bacteria. These are protein complexes, where multiple individual enzymes interact to form the active PKS. Each individual enzyme unit possess KS, CLF, or ACP activity. Type II PKS form non-reduced polyketides that spontaneously fold into complex aromatic/cyclic/polycylic compound. Folding of the polyketide backbones is most often assisted/directed by different classes of enzymes called aromatases and cyclases that act independently of the PKS enzyme to promote a non-spontaneous folding reaction. The biosynthesis of a polyaromatic compound in these systems typically involves the successive action of multiple different aromatases/cyclases, which can be divided into two groups based on which types of substrates they act on: the first acts on linear polyketide chains to catalyze the formation of the first aromatic/cyclic group, while the second only accepts substrates that already contain aromatic/cyclic groups, i.e. products from the first group.

Type III PKS have been found in bacteria, fungi, and plants. They typically consist of only a KS domain, which is usually referred to as a KASIII or a chalcone synthase domain. This KS domain acts independently of the ACP domain. The products of Type III PKS often spontaneously fold into complex aromatic/cyclic/polycyclic compounds. They are self-contained enzymes that form homodimers. Their single active site in each monomer catalyzes the priming, extension, and cyclization reactions iteratively to form polyketide products.

Functional PKS active units can be formed by combining different modules from one or more of the type classes described above. Varied combinations of different KS and one or more ACP, AT, SAT, CYC, KR, DH, ER, and/or Cmet module types, with each included module type represented by single or multiple modules, can generate a functional PKS active unit—making possible a multitude of varied polyketide products.

In some embodiments, the KS, CLF, ACP, and AT steps are performed by Type I, II or III PKS enzymes or a portion thereof and producing an octaketide. In some embodiments, the PKS enzyme comprises an amino acid sequence (or catalytic portion thereof) selected from SEQ ID NO:10 (of Aloe arborescens, SEQ ID NO:11 (of Hypericum perforatum), SEQ ID NO:3 (of Streptomyces spp.), SEQ ID NO:4 (of Streptomyces spp.), SEQ ID NO:5 (of Streptomyces spp.), SEQ ID NO:6 (of Saccharomyces cerevisiae), SEQ ID NO:7 (of Schizosaccharomyces pombe), SEQ ID NO:8 (of Yarrowia lipolytica), and/or SEQ ID NO:9 (Escherichia coli). In some embodiments, the enzyme comprises an amino acid sequence (or catalytic portion thereof) selected from SEQ ID NO:3 or 4 (of Streptomyces coelicolor) or SEQ ID NO:5 (of Streptomyces sp. R1128). In some embodiments, at least one PKS, KS, CLF, ACP or AT enzyme is an engineered variant of any one of SEQ ID NOS: 3, 4, 5, 6, 7, 8, 9, 10 and 11 (or catalytic portion thereof). An engineered variant can generally comprise an amino acid sequence having from 1 to 50, or from 1 to 20, or from 1 to 10 amino acid modifications independently selected from substitutions, insertions, or deletions. In some embodiments, the engineered variant is at least 50% identical, or at least 75% identical, or at least 90% identical, or at least 95% identical, or at least 98% identical to the parent enzyme.

In some embodiments, the CYC steps convert the octaketide to the cyclized FK product. In some embodiments, the CYC steps are mediated by one or more enzymes from Streptomyces spp. In some embodiments, the enzyme comprises the amino acid sequence of an enzyme from Streptomyces sp. R1128. In some embodiments, the enzyme comprises the amino acid sequence of SEQ ID NO: 12 (ZhuI) or SEQ ID NO:13 (ZhuJ), or catalytic portion thereof. In some embodiments, at least one CYC enzyme is an engineered variant of SEQ ID NO:12 or SEQ ID NO:13, or catalytic portion thereof. An engineered variant can generally comprise from 1 to 50, or from 1 to 20, or from 1 to 10 amino acid modifications independently selected from substitutions, insertions, or deletion. In some embodiments, the engineered variant is at least 50% identical, or at least 75% identical, or at least 90% identical, or at least 95% identical, or at least 98% identical to the parent enzyme.

One or more monooxygenase enzymes convert FKA to CA, through flavokermesic acid (FA). In some embodiments, these steps are performed by different monooxygenase enzymes (shown as MO1 and MO2 in the pathway in FIG. 2). In various embodiments, one or both of these enzymes are CYP450 enzymes. In some embodiments, one or both of these enzymes are laccases. In some embodiments, one or both of these enzymes are non-heme iron oxygenases (NHIO). In some embodiments, the MO1 and/or MPO2 are selected based on a library screen of CYP450s, laccases, and/or NHIOs. In some embodiments, a monooxygenase is an insect enzyme, optionally selected from Dactyopius coccus, Coccus hesperidum, Porphyrophora polonica, Porphyrophora hamelii, Palmicultor browni, or Pseudococcus longispinus. In some embodiments, at least one MO enzyme is an engineered variant. An engineered variant can generally comprise from 1 to 50, or from 1 to 20, or from 1 to 10 amino acid modifications independently selected from substitutions, insertions, or deletion. In some embodiments, the engineered variant is at least 50% identical, or at least 75% identical, or at least 90% identical, or at least 95% identical, or at least 98% identical to the parent enzyme.

C-UGT or C-glucosyltransferase, glucosylates the 2-carbon on either flavokermesic acid (FA) or kermesic acid (KA). This enzyme is expressed in the cochineal bug Dactylopius coccus. Kannangara, R et al., Characterization of a membrane-bound C-glucosyltransferase responsible for carminic acid biosynthesis in Dactylopius coccus Costa, Nature Communication 8:1987 (2017); and WO 2015/091843, which are hereby incorporated by reference in their entireties. The nucleotide sequence for C-UGT is provided as SEQ ID NO:2 (GenBank: KY860725.1). The amino acid sequence is SEQ ID NO:2 (ATL15304.1). In some embodiments, the C-UGT is an engineered variant. An engineered variant can generally comprise from 1 to 50, or from 1 to 20, or from 1 to 10 amino acid modifications independently selected from substitutions, insertions, or deletion. In some embodiments, the engineered variant is at least 50% identical, or at least 75% identical, or at least 90% identical, or at least 95% identical, or at least 98% identical to the parent enzyme.

Other aspects and embodiments of the invention will be apparent from this detailed description.

All patents and publications referenced herein are hereby incorporated by reference in their entireties.

SEQUENCES

SEQ ID NO: 1
>Carminic Acid C-UCT nucleotide sequence (CenBank: KY860725.1)
ATGGAATTTCGTTTACTAATCCTGGCTCTTTTTTCTGTACTTATGAGTACTTCAAACGGAGCAGAAATTTTAGCTCTTTT
CCCTATTCACGGTATCAGTAATTATAATGTTGCTGAAGCACTGCTGAAGACCTTAGCTAACCGGGGTCATAATGTTACAG
TTGTCACATCTTTTCCTCAAAAAAAACCTGTACCTAATTTGTACGAAATTGACGTATCTGGAGCTAAAGGCTTGGCTACT
AATTCAATACATTTTGAAAGATTACAAACGATTATTCAAGATGTAAAATCGAACTTTAAGAACATGGTACGACTTAGCAG
AACATACTGTGAGATTATGTTTTCTGATCCGAGGGTTTTGAACATTCGAGACAAGAAATTCGATCTCGTAATAAACGCCG
TATTTGGCAGTGACTGCGATGCCGGATTCGCATGGAAAAGTCAAGCTCCATTGATTTCAATTCTCAATGCTAGACATACT
CCTTGGGCCCTACACAGAATGGGAAATCCATCAAATCCAGCGTATATGCCTGTCATTCATTCTAGATTTCCTGTAAAAAT
GAATTTCTTCCAAAGAATGATAAATACGGGTTGGCATTTGTATTTTCTGTACATGTACTTTTATTATGGTAATGGAGAAC
ATCCCAACAAAATGCCCAGAAAATTTTTTCGCAACCACATCCCCGACATAAATCAAATGGTTTTTAATACATCTTTATTA
TTCGTAAATACTCACTTTTCGGTTGATATGCCATATCCTTTGGTTCCAAACTGCATTGAAATAGGAGGAATACATGTAAA
AGAGCCACAACCACTGCCTTTGGAAATACAAAAATTCATGGACGAAGCAGAACATGGGGTCATTTTCTTCACGCTAGGAT
CAATGGTGCGTACTTCCACGTTTCCAAATCAAACTATTCAAGCATTTAAGGAAGCTTTTGCCGAATTACCTCAAAGAGTC
TTATGGAAGTTTGAGAATGAAAATGAGGATATGCCATCAAATGTACTCATAAGGAAATGGTTTCCACAAAATGATATATT
CGGTCATAAGAATATCAAAGCATTCATTAGTCACGGTGGAAATTCTGGAGCTCTGGAGGCTGTTCATTTCGGAGTACCGA
TAATTGGAATTCCTTTATTCTACGATCAGTACAGGAATATTTTGAGTTTCGTTAAAGAAGGTGTTGCCGTTCTTTTGGAT
GTGAATGATCTGACGAAAGATAATATTTTATCTTCTGTCAGGACTGTTGTTAATGATAAGAGTTACTCAGAACGTATGAA
ACCATTGTCACAACTATTCCCAGATCCACCAATCAGTCCTCTTCACACACCTGTTTACTGGACAGAATATGTCATCCGCC
ATAGAGGAGCCCATCACCTCAAGACCGCTGGCGCATTTTTGCATTGGTATCAGTATTTACTTTTGGACGTTATTACCTTC
TTATTAGTCACATTCTCCGCTTTTTGTTTTATTGTGAAATATATATCTAAAGCTCTCATTCATCATTATTGGAGCAGTTC
GAAATCTGAAAAGTTGAAAAAAAATTAA
SEQ ID NO: 2
>Carminic Acid C-UGT amino acid sequence (GenBank: ATL15304.1)
MEFRLLILALFSVLMSTSNGAEILALFPIHGISNYNVAEALLKTLANRGHNVTVVTSFPQKKPVPNLYEIDVSGAKGLAT
NSIHFERLQTIIQDVKSNFKNMVRLSRTYCEIMFSDPRVLNIRDKKFDLVINAVFGSDCDAGFAWKSQAPLISILNARHT
PWALHRMGNPSNPAYMPVIHSRFPVKMNFFQRMINTGWHLYFLYMYFYYGNGEDANKMARKFFGNDMPDINEMVFNTSLL
FVNTHFSVDMPYPLVPNCIEIGGIHVKEPQPLPLEIQKFMDEAEHGVIFFTLGSMVRTSTFPNQTIQAFKEAFAELPQRV
LWKFENENEDMPSNVLIRKWFPQNDIFGHKNIKAFISHGGNSGALEAVHFGVPIIGIPLFYDQYRNILSFVKEGVAVLLD
VNDLTKDNILSSVRTVVNDKSYSERMKALSQLFRDRPMSPLDTAVYWTEYVIRHRGAHHLKTAGAFLHWYQYLLLDVITF
LLVTFCAFCFIVKYICKALIHHYWSSSKSEKLKKN
SEQ ID NO: 3
>Streptomyces coelicolor KS1 amino acid sequence (Q02059)
MPLDAAPVDPASRGPVSAFEPPSSHGADDDDDHRTNASKELFGLKRRVVITGVGVRAPGGNGTRQFWELLTSGRTATRRI
SFFDPSPYRSQVAAEADFDPVAEGFGPRELDRMDRASQFAVACAREAFAASGLDPDTLDPARVGVSLGSAVAAATSLERE
YLLLSDSGRDWEVDAAWLSRHMFDYLVPSVMPAEVAWAVGAEGPVTMVSTGCTSGLDSVGNAVRAIEEGSADVMFAGAAD
TPITPIVVACFDAIRATTARNDDPEHASRPFDGTRDGFVLAEGAAMFVLEDYDSALARGARIHAEISGYATRCNAYHMTG
LKADGREMAETIRVALDESRTDATDIDYINAHGSGTRQNDRHETAAYKRALGEHARRTPVSSIKSMVGHSLGAIGSLEIA
ACVLALEHGVVPPTANLRTSDPECDLDYVPLEARERKLRSVLTVGSGFGGFQSAMVLRDAETAGAAA
SEQ ID NO: 4
>Streptomyces coelicolor KS2 (CLF) amino acid sequence (Q02062)
MSVLITGVGVVAPNGLGLAPYWSAVLDGRHGLGPVTRFDVSRYPATLAGQIDDFHAPDHIPGRLLPQTDPSTRLALTAAD
WALQDAKADPESLTDYDMGVVTANACGGFDFTHREFRKLWSEGPKSVSVYESFAWFYAVNTGQISIRHGMRGPSSALVAE
QAGGLDALGHARRTIRRGTPLVVSGGVDSALDPWGWVSQIASGRISTATDPDRAYLPFDERAAGYVPGEGGAILVLEDSA
AAEARGRHDAYGELAGCASTFDPAPGSGRPAGLERAIRLALNDAGTGPEDVDVVFADGAGVPELDAAEARAIGRVFGREG
VPVTVPKTTTGRLYSGGGPLDVVTALMSLREGVIAPTAGVTSVPREYGIDLVLGEPRSTAPRTALVLARGRWGFNSAAVL
RRFAPTP
SEQ ID NO: 5
>Streptomyces sp. R1128 zhuN (ACP) amino acid sequence (Q9F6C8)
MTIDDLRRILTECAGEDESVDLGGDILDTPFTELGYDSLALMETAARIEQEFGVAIPDDEFAELATPRAVLAAVSTAVSA
AA
SEQ ID NO: 6
>Saccharomyces cerevisiae FAS1 (AT) amino acid sequence (P07149)
MDAYSTRPLTLSHGSLEHVLLVPTASFFIASQLQEQFNKILPEPTEGFAADDEPTTPAELVGKFLGYVSSLVEPSKVGQF
DQVLNLCLTEFENCYLEGNDIHALAAKLLQENDTTLVKTKELIKNYITARIMAKRPFDKKSNSALFRAVGEGNAQLVAIF
GGQGNTDDYFFELRDLYQTYHVLVGDLIKFSAETLSELIRTTLDAEKVFTQGLNILEWLENPSNTPDKDYLLSIPISCPL
IGVIQLAHYVVTAKLLGFTPGELRSYLKGATGHSQGLVTAVAIAETDSWESFFVSVRKAITVLFFIGVRCYEAYPNTSLP
PSILEDSLENNEGVPSPMLSISNLTQEQVQDYVNKTNSHLPAGKQVEISLVNGAKNLVVSGPPQSLYGLNLTLRKAKAPS
GLDQSRIPFSERKLKFSNRFLPVASPFHSHLLVPASDLINKDLVKNNVSFNAKDIQIPVYDTFDGSDLRVLSGSISERIV
DCIIRLPVKWETTTQFKATHILDFGPGGASGLGVLTHRNKDGTGVRVIVAGTLDINPDDDYGFKQEIFDVTSNGLKKNPN
WLEEYHPKLIKNKSGKIFVETKFSKLIGRPPLLVPGMTPCTVSPDFVAATTNAGYTIELAGGGYFSAAGMTAAIDSVVSQ
IEKGSTFGINLIYVNPFMLQWGIPLIKELRSKGYPIQFLTIGAGVPSLEVASEYIETLGLKYLGLKPGSIDAISQVINLA
KAHPNFPIALQWTGGRGGGHHSFEDAHTPMLQMYSKIRRHPNIMLIFGSGFGSADDTYPYLTGEWSTKFDYPPMPFDGFL
FGSRVMIAKEVKTSPDAKKCIAACTGVPDDKWEQTYKKPTGGIVTVRSEMGEPIHKIATRGVMLWKEFDETIFNLPKNKL
VPTLEAKRDYIISRLNADFQKPWFATVNGQARDLATMTYEEVAKRLVELMFIRSTNSWFDVTWRTFTGDFLRRVEERFTK
SKTLSLIQSYSLLDKPDEAIEKVFNAYPAAREQFLNAQDIDHFLSMCQNPMQKPVPFVPVLDRRFEIFFKKDSLWQSEHL
EAVVDQDVQRTCILHGPVAAQFTKVIDEPIKSIMDGIHDGHIKKLLHQYYGDDESKIPAVEYFGGESPVDVQSQVDSSSV
SEDSAVFKATSSTDEESWFKALAGSEINWRHASFLCSFITQDKMFVSNPIRKVFKPSQGMVVEISNGNTSSKTVVTLSEP
VQGELKPTVILKLLKENIIQMEMIENRTMDGKPVSLPLLYNFNPDNGFAPISEVMEDRNQRIKEMYWKLWIDEPFNLDFD
PRDVIKGKDFEITAKEVYDFTHAVGNNCEDFVSRPDRTMLAPMDFAIVVGWRAIIKAIFPNTVDGDLLKLVHLSNGYKMI
PGAKPLQVGDVVSTTAVIESVVNQPTGKIVDVVGTLSRNGKPVMEVTSSFFYRGNYTDFENTFQKTVEPVYQMHIKTSKD
IAVLRSKEWFQLDDEDFDLLNKTLTFETETEVTFKNANIFSSVKCFGPIKVELPTKETVEIGIVDYEAGASHGNPVVDFL
KRNGSTLEQKVNLENPIPIAVLDSYTPSTNEPYARVSGDLNPIHVSRHFASYANLPGTITHGMFSSASVRALIENWAADS
VSSRVRGYTCQFVDMVLPNTALKTSIQHVGMINGRKLIKFETRNEDDVVVLTGEAEIEQPVTTFVFTGQGSQEQGMGMDL
YKTSKAAQDVWNRADNHFKDTYGFSILDIVINNPVNLTIHFGGEKGKRIRENYSAMIFETIVDGKLKTEKIFKEINEHST
SYTFRSEKGLLSATQFTQPALTLMEKAAFEDLKSKGLIPADATFAGHSLGEYAALASLADVMSIESLVEVVFYRGMTMQV
AVPRDELGRSNYGMIAINPGRVAASFSQEALQYVVERVGKRTGWLVEIVNYNVENQQYVAAGDLRALDTVINVLNFIKLQ
KIDIIELQKSLSLEEVEGHLFEIIDEASKKSAVKPRPLKLERGFACIPLVGISVPFHSTYLMNGVKPFKSFLKKNIIKEN
VKVARLAGKYIPNLTAKPFQVTKEYFQDVYDLIGSEPIKEIIDNWEKYEQS
SEQ ID NO: 7
>Schizosaccharomyces pombe FAS1 (AT) amino acid sequence (Q9UUG0)
MVEAEQVHQSLRSLVLSYAHFSPSILIPASQYLLAAQLRDEFLSLHPAPSAESVEKEGAELEFEHELHLLAGFLGLIAAK
EEETPGQYTQLLRIITLEFERTFLAGNEVHAVVHSLGLNIPAQKDVVRFYYHSCALIGQTTKFHGSALLDESSVKLAAIF
GGQGYEDYFDELIELYEVYAPFAAELIQVLSKHLFTLSQNEQASKVYSKGLNVLDWLAGERPERDYLVSAPVSLPLVGLT
QLVHFSVTAQILGLNPGELASRFSAASGHSQGIVVAAAVSASTDSASFMENAKVALTTLFWIGVRSQQTFPTTTLPPSVV
ADSLASSEGNPTPMLAVRDLPIETLNKHIETTNTHLPEDRKVSLSLVNGPRSFVVSGPARSLYGLNLSLRKEKADGQNQS
RIPHSKRKLRFINRFLSISVPFHSPYLAPVRSLLEKDLQGLQFSALKVPVYSTDDAGDLRFEQPSKLLLALAVMITEKVV
HWEEACGFPDVTHIIDFGPGGISGVGSLTRANKDGQGVRVIVADSFESLDMGAKFEIFDRDAKSIEFAPNWVKLYSPKLV
KNKLGRVYVDTRLSRMLGLPPLWVAGMTPTSVPWQFCSAIAKAGFTYELAGGGYFDPKMMREAIHKLSLNIPPGAGICVN
VIYINPRTYAWQIPLIRDMVAEGYPIRGVTIAAGIPSLEVANELISTLGVQYLCLKPGSVEAVNAVISIAKANPTFPIVL
QWTGGRAGGHHSFEDFHSPILLTYSAIRRCDNIVLIAGSGFGGADDTEPYLIGEWSAAFKLPPMPFDGILFGSRLMVAKE
AHTSLAAKEAIVAAKGVDDSEWEKTYDGPIGGIVTVLSELGEPIHKLATRGIMFWKELDDTIFSLPRPKRLPALLAKKQY
IIKRLNDDFQKVYFPAHIVEQVSPEKFKFEAVDSVEDMTYAELLYRAIDLMYVTKEKRWIDVTLRTFTGKLMRRIEERFT
QDVGKTTLIENFEDLNDPYPVAARFLDAYPEASTQDLNTQDAQFFYSLCSNPFQKPVPFIPAIDDTFEFYFKKDSLWQSE
DLAAVVGEDVGRVAILQGPMAAKHSTKVNEPAKELLDGINETHIQHFIKKFYAGDEKKIPIVEYFGGVPPVNVSHKSLES
VSVTEEAGSKVYKLPEIGSNSALPSKKLWFELLAGPEYTWFRAIFTTQRVAKGWKLEHNPVRRIFAPRYGQRAVVKGKDN
DTVVELYETQSGNYVLAARLSYDGETIVVSMFENRNALKKEVHLDFLFKYEPSAGYSPVSEILDGRNDRIKHFYWALWFG
EEPYPENASITDTFTGPEVTVTGNMIEDFCRTVGNHNEAYTKRAIRKRMAPMDFAIVVGWQAITKAIFPKAIDGDLLRLV
HLSNSFRMVGSHSLMEGDKVTTSASIIAILNNDSGKTVTVKGTVYRDGKEVIEVISRFLYRGTFTDFENTFEHTQETPMQ
LTLATPKDVAVLQSKSWFQLLDPSQDLSGSILTFRLNSYVRFKDQKVKSSVETKGIVLSELPSKAIIQVASVDFQSVDCH
GNPVIEFLKRNGKPIEQPVEFENGGYSVIQVMDEGYSPVFVTPPTNSPYAEVSGDYNPIHVSPTFAAFVELPGTHGITHG
MYTSAAARRFVETYAAQNVPERVKHYEVTFVNMVLPNTELITKLSHTGMINGRKIIKVEVLNQETSEPVLVGTAEVEQPV
SAYVFTGQGSQEQGMGMDLYASSPVARKIWDSADKHFLTNYGFSIIDIVKHNPHSITIHFGGSKGKKIRDNYMAMAYEKL
MEDGTSKVVPVFETITKDSTSFSFTHPSGLLSATQFTQPALTLMEKSAFEDMRSKGLVQNDCAFAGHSLGEYSALSAMGD
VLSIEALVDLVFLRGLTMQNAVHRDELGRSDYGMVAANPSRVSASFTDAALRFIVDHIGQQTNLLLEIVNYNVENQQYVV
SGNLLSLSTLGHVLNFLKVQKIDFEKLKETLTIEQLKEQLTDIVEACHAKTLEQQKKTGRIELERGYATIPLKIDVPFHS
SFLRGGVRMFREYLVKKIFPHQINVAKLRGKYIPNLTAKPFEISKEYFQNVYDLTGSQRIKKILQNWDEYESS
SEQ ID NO: 8
>Yarrowia lipolytica FAS1 (AT) amino acid sequence (P34229)
MYPTTGVNTPQSAASLRPLVLSHGQTEHSLLVPTSLYINCTTLRDQFYASLPPATEDKADDDEPSSSTELLAAFLGFTAK
TVEEEPGPYDDVLSLVLNEFETRYLRGNDIHAVASSLLQDEDVPTTVGKIKRVIRAYYAARIACNRPIKAHSSALFRAAS
EDSDNVSLYAIFGGQGNTEDYFEELREIYDIYQGLVGDFIRECGAQLLALSRDHIAAEKIYTKGFDIVKWLEHPETIPDF
EYLISAPISVPIIGVIQLAHYAVTCRVLGLNPGQVRDNLKGATGHSQGLITAIAISASDSWDEFYNSASRILKIFFFIGV
RVQQAYPSTFLPPSTLEDSVKQGEGKPTPMLSIRDLSLNQVQEFVDATNLHLPEDKQIVVSLINGPRNVVVTGPPQSLYG
LCLVLRKQKAETGLDQSRVPHSQRKLKFTHRFLPITSPFHSYLLEKSTDLIINDLESSGVEFVSSELKVPVYDTFDGSVL
SQLPKGIVSRLVNLITHLPVKWEKATQFQASHIVDFGPGGASGLGLLTHKNKDGTGVRTILAGVIDQPLEFGFKQELFDR
QESSIVFAQNWAKEFSPKLVKISSTNEVYVDTKFSRLTGRAPIMVAGMTPTTVNPKFVAATMNSGYHIELGGGGYFAPGM
MTKALEHIEKNTPPGSGITINLIYVNPRLIQWGIPLIQELRQKGFPIEGLTIGAGVPSLEVANEWIQDLGVKHIAFKPGS
IEAISSVIRIAKANPDFPIILQWTGGRGGGHHSFEDFHAPILQMYSKIRRCSNIVLIAGSGFGASTDSYPYLTGSWSRDF
DYPPMPFDGILVGSRVMVAKEAFTSLGAKQLIVDSPGVEDSEWEKTYDKPTGGVITVLSEMGEPIHKLATRGVLFWHEMD
KTVFSLPKKKRLEVLKSKRAYIIKRLNDDFQKTWFAKNAQGQVCDLEDLTYAEVIQRLVDLMYVKKESRWIDVTLRNLAG
TFIRRVEERFSTETGASSVLQSFSELDSEPEKVVERVFELFPASTTQIINAQDKDHFLMLCLNPMQKPVPFIPVLDDNFE
FFFKKDSLWQCEDLAAVVDEDVGRICILQGPVAVKHSKIVNEPVKEILDSMHEGHIKQLLEDGEYAGNMANIPQVECFGG
KPAQNFGDVALDSVMVLDDLNKTVFKIETGTSALPSAADWFSLLAGDKNSWRQVFLSTDTIVQTTKMISNPLHRLLEPIA
GLQVEIEHPDEPENTVISAFEPINGKVTKVLELRKGAGDVISLQLIEARGVDRVPVALPLEFKYQPQIGYAPIVEVMTDR
NTRIKEFYWKLWFGQDSKFEIDTDITEEIIGDDVTISGKAIADFVHAVGNKGEAFVGRSTSAGTVFAPMDFAIVLGWKAI
IKAIFPRAIDADILRLVHLSNGFKMMPGADPLQMGDVVSATAKIDTVKNSATGKTVAVRGLLTRDGKPVMEVVSEFFYRG
EFSDFQNTFERREEVPMQLTLKDAKAVAILCSKEWFEYNGDDTKDLEGKTIVFRNSSFIKYKNETVFSSVHTTGKVLMEL
PSKEVIEIATVNYQAGESHGNPVIDYLERNGTTIEQPVEFEKPIPLSKADDLLSFKAPSSNEPYAGVSGDYNPIHVSRAF
ASYASLPGTITHGMYSSAAVRSLIEVWAAENNVSRVRAFSCQFQGMVLPNDEIVTRLEHVGMINGRKIIKVISTNRETEA
VVLSGEAEVEQPISTFVFTGQGSQEQGMGMDLYASSEVAKKVWDKADEHFLQNYGFSIIKIVVENPKELDIHFGGPKGKK
IRDNYISMMFETIDEKTGNLISEKIFKEIDETTDSFTFKSPTGLLSATQFTQPALTLMEKASFEDMKAKGLVPVDATFAG
HSLGEYSALASLGDVMPIESLVDVVFYRGMTMQVAVPRDAQGRSNYGMCAVNPSRISTTFNDAALRFVVDHISEQTKWLL
EIVNYNVENSQYVTAGDLRALDTLTNVLNVLKLEKINIDKLLESLPLEKVKEHLSEIVTEVAKKSVAKPQPIELERGFAV
IPLKGISVPFHSSYLRNGVKPFQNFLVKKVPKNAVKPANLIGKYIPNLTAKPFEITKEYFEEVYKLTGSEKVKSIINNWE
SYESKQ
SEQ ID NO: 9
>Escherichia coli FABH (AT) amino acid sequence (P0A6R0)
MYTKIIGTGSYLPEQVRTNADLEKMVDTSDEWIVTRTGIRERHIAAPNETVSTMGFEAATRAIEMAGIEKDQIGLIVVAT
TSATHAFPSAACQIQSMLGIKGCPAFDVAAACAGFTYALSVADQYVKSGAVKYALVVGSDVLARTCDPTDRGTIIIFGDG
AGAAVLAASEEPGIISTHLHADGSYGELLTLPNADRVNPENSIHLTMAGNEVFKVAVTELAHIVDETLAANNLDRSQLDW
LVPHQANLRIISATAKKLGMSMDNVVVTLDRHGNTSAASVPCALDEAVRDGRIKPGQLVLLEAFGGGFTWGSALVRF
SEQ ID NO: 10
>Aloe arborescens PKS amino acid sequence (AAT48709)
MSSLSNASHLMEDVQGIRKAQRADGTATVMAIGTAHPPHIFPQDTYADFYFRATNSEHKVELKKKFDRICKKTMIGKRYF
NYDEEFLKKYPNITSFDEPSLNDRQDICVPGVPALGAEAAVKAIAEWGRPKSEITHLVFCTSCGVDMPSADFQCAKLLGL
RTNVNKYCVYMQGCYAGGTVMRYAKDLAENNRGARVLVVCAELTIIGLRGPNESHLDNAIGNSLFGDGAAALIVGSDPII
GVEKPMFEIVCAKQTVIPNSEDVIHLHMREAGLMFYMSKDSPETISNNVEACLVDVFKSVGMTPPEDWNSLFWIPHPGGR
AILDQVEAKLKLRPEKFRATRTVLWDCGNMVSACVLYILDEMRRKSADEGLETYGEGLEWGVLLGFGPGMTVETILLHSL
PLM
SEQ ID NO: 11
>Hypericum perforatum PKS amino acid sequence (AEE69029)
MGSLDNGSARINNQKSNGLASILAIGTALPPICIKQDDYPDYYFRVTKSDHKTQLKEKFRRICEKSGVTKRYTVLTEDMI
KENENIITYKAPSLDARQAILHKETPKLAIEAALKTIQEWGQPVSKITHLFFCSSSGGCYLPSSDFQIAKALGLEPTVQR
SMVFPHGCYAASSGLRLAKDIAENNKDARVLVVCCELMVSSFHAPSEDAIGMLIGHAIFGDGAACAIVGADPGPTERPIF
ELVKGGQVIVPDTEDCLGGWVMEMGWIYDLNKRLPQALADNILGALDDTLRLTGKRDDLNGLFYVLHPGGRAIIDLLEEK
LELTKDKLESSRRVLSNYGNMWGPALVFTLDEMRRKSKEDNATTTGGGSELGLMMAFGPGLTTEIMVLRSVPL
SEQ ID NO: 12
>Streptomyces sp. R1128 ZhuI (CYC) amino acid sequence (Q9F6D3)
MRHVEHTVTVAAPADLVWEVLADVLGYADIFPPTEKVEILEEGQGYQVVRLHVDVAGEINTWTSRRDLDPARRVIAYRQL
ETAPIVGHMSGEWRAFTLDAERTQLVLTHDFVTRAAGDDGLVAGKLTPDEAREMLEAVVERNSVADLNAVLGEAERRVRA
AGGVGTVTA
SEQ ID NO: 13
>Streptomyces sp. R1128 ZhuJ (CYC) amino acid sequence (Q9F6D2)
MSGRKTFLDLSFATRDTPSEATPVVVDLLDHVTGATVLGLSPEDFPDGMAISNETVTLTTHTGTHMDAPLHYGPLSGGVP
AKSIDQVPLEWCYGPGVRLDVRHVPAGDGITVDHLNAALDAAEHDLAPGDIVMLWTGADALWGTREYLSTFPGLTGKGTQ
FLVEAGVKVIGIDAWGLDRPMAAMIEEYRRTGDKGALWPAHVYGRTREYLQLEKLNNLGALPGATGYDISCFPVAVAGTG
AGWTRVVAVFEQEEED

What is claimed is:

1. A host cell for producing carminic acid, the host cell expressing an enzymatic pathway for biosynthesis of carminic acid from polyketide building blocks.

2. The host cell of claim 1, wherein the host cell is a yeast or bacteria.

3. The host cell of claim 2, wherein the host cell is a species of Saccharomyces, Pichia, or Yarrowia, which is optionally Saccharomyces cerevisiae, Pichia pastoris, and Yarrowia lipolytica.

4. (canceled)

5. The host cell of claim 2, wherein the host cell is a bacteria selected from Escherichia spp., Bacillus spp Corynebacterium spp Rhodobacter spp Zymomonas spp Vibrio spp., and Pseudomonas spp., and which is optionally Escherichia coli, Bacillus subtilis, Corynebacterium glutamicum, Rhodobacter capsulatus, Rhodobacter sphaeroides, Zymomonas mobilis, Vibrio natriegens, or Pseudomonas putida.

6. (canceled)

7. The host cell of claim 1, wherein the host cell expresses:
a recombinant fatty acid synthase (FAS)/polyketide synthase (PKS) that converts Acetyl-CoA and/or Malonyl-CoA building blocks to flavokermesic anthrone (FKA);
a monooxygenase enzyme that converts FKA to flavokermesic acid (FK), and a monooxygenase enzyme that converts FK to kermesic acid (KA), where the monooxygenases can be the same or different; and
a C-UDP-glycosyltransferase (C-UGT) that glycosylates FK and/or KA substrate.

8. The host cell of claim 1, wherein the host cell expresses one or more enzymes of a bacteria, fungus, plant or insect species, or an engineered variant thereof.

9. The host cell of claim 8, wherein the host cell expresses one or more enzymes of Dactyopius coccus, Coccus hesperidum, Porphyrophora polonica, Porphyrophora hamelii, Palmicultor browni, or Pseudococcus longispinus.

10. The host cell of claim 8, wherein the host cell expresses one or more enzymes of Aloe arborescens, Hypericum perforatum, Streptomyces spp., Saccharomyces cerevisiae, Schizosaccharomyces pombe, Yarrowia lipolytica, and Escherichia coli.

11. The host cell of claim 10, wherein the host cell expresses one or more enzymes of Streptomyces coelicolor or Streptomyces sp. R1128.

12. The host cell of claim 9, wherein the FAS/PKS enzyme is an insect enzyme or engineered variant thereof, wherein the FAS/PKS enzyme is optionally an enzyme of Dactyopius coccus, Coccus hesperidum, Porphyrophora polonica, Porphyrophora hamelii, Palmicultor browni, or Pseudococcus longispinus, or an engineered variant thereof.

13. The host cell of claim 8, wherein the PKS enzyme is a plant, fungal, or bacterial enzyme that possesses the octaketide synthase and cyclase activities.

14. The host cell of claim 13, wherein the FAS/PKS enzyme comprises an enzyme of Aloe arborescens, Hypericum perforatum, Streptomyces spp., Saccharomyces cerevisiae, Schizosaccharomyces pombe, Yarrowia lipolytica, or Escherichia coli, or a catalytically active portion or derivative thereof.

15. The host cell of claim 14, wherein the FAS/PKS enzyme comprises an enzyme of Streptomyces coelicolor or Streptomyces sp. R1128, or a catalytically active portion or derivative thereof.

16. The host cell of claim 7, wherein modules of Type I and Type II polyketide synthases are assembled to create a polyketide synthase system capable of flavokermesic acid anthrone or flavokermesic acid biosynthesis.

17. The host cell of claim 13, wherein the PKS enzyme comprises an amino acid sequence selected from SEQ ID NO:10, SEQ ID NO:11, SEQ ID NO:3, SEQ ID NO:4, SEQ ID NO:5, SEQ ID NO:6, SEQ ID NO:7, SEQ ID NO:8, SEQ ID NO:9, SEQ ID NO: 12 and/or SEQ ID NO:13, or a catalytic portion and/or engineered variant thereof.

18. (canceled)

19. The host cell of claim 1, wherein a single monooxygenase enzyme converts FKA to CA, through flavokermesic acid (FK).

20. The host cell of claim 1, wherein a first monooxygenase enzyme converts FKA to FK, and a second monooxygenase enzyme converts FK to CA.

21-23. (canceled)

24. The host cell of claim 19, wherein one or more monooxygenase enzymes is an insect enzyme, optionally selected from Dactyopius coccus, Coccus hesperidum, Porphyrophora polonica, Porphyrophora hamelii, Palmicultor browni, or Pseudococcus longispinus; or is an engineered variant thereof.

25. The host cell of claim 1, wherein the C-UGT comprises the amino acid sequence of SEQ ID NO:2, or an engineered variant thereof.

26. A method for producing carminic acid, comprising, culturing the microbial cell of claim 1 under conditions suitable for producing carminic acid.

27. (canceled)


SEQUENCE LISTING (13 sequences)

SEQ ID NO: 1 - DNA, 1548 bp, Dactylopius coccus
atggaatttcgtttactaatcctggctcttttttctgtacttatgagtacttcaaacggagcagaaattttagctcttttccctattcacggtatcagtaattataatgttgctgaagcactgctgaagaccttagctaaccggggtcataatgttacagttgtcacatcttttcctcaaaaaaaacctgtacctaatttgtacgaaattgacgtatctggagctaaaggcttggctactaattcaatacattttgaaagattacaaacgattattcaagatgtaaaatcgaactttaagaacatggtacgacttagcagaacatactgtgagattatgttttctgatccgagggttttgaacattcgagacaagaaattcgatctcgtaataaacgccgtatttggcagtgactgcgatgccggattcgcatggaaaagtcaagctccattgatttcaattctcaatgctagacatactccttgggccctacacagaatgggaaatccatcaaatccagcgtatatgcctgtcattcattctagatttcctgtaaaaatgaatttcttccaaagaatgataaatacgggttggcatttgtattttctgtacatgtacttttattatggtaatggagaagatgccaacaaaatggcgagaaaattttttggcaacgacatgcccgacataaatgaaatggtttttaatacatctttattattcgtaaatactcacttttcggttgatatgccatatcctttggttccaaactgcattgaaataggaggaatacatgtaaaagagccacaaccactgcctttggaaatacaaaaattcatggacgaagcagaacatggggtcattttcttcacgctaggatcaatggtgcgtacttccacgtttccaaatcaaactattcaagcatttaaggaagcttttgccgaattacctcaaagagtcttatggaagtttgagaatgaaaatgaggatatgccatcaaatgtactcataaggaaatggtttccacaaaatgatatattcggtcataagaatatcaaagcattcattagtcacggtggaaattctggagctctggaggctgttcatttcggagtaccgataattggaattcctttattctacgatcagtacaggaatattttgagtttcgttaaagaaggtgttgccgttcttttggatgtgaatgatctgacgaaagataatattttatcttctgtcaggactgttgttaatgataagagttactcagaacgtatgaaagcattgtcacaactattccgagatcgaccaatgagtcctcttgacacagctgtttactggacagaatatgtcatccgccatagaggagcccatcacctcaagaccgctggcgcatttttgcattggtatcagtatttacttttggacgttattaccttcttattagtcacattctgcgctttttgttttattgtgaaatatatatgtaaagctctcattcatcattattggagcagttcgaaatctgaaaagttgaaaaaaaattaa

SEQ ID NO: 2 - protein, 515 aa, Dactylopius coccus
MEFRLLILALFSVLMSTSNGAEILALFPIHGISNYNVAEALLKTLANRGHNVTVVTSFPQKKPVPNLYEIDVSGAKGLATNSIHFERLQTIIQDVKSNFKNMVRLSRTYCEIMFSDPRVLNIRDKKFDLVINAVFGSDCDAGFAWKSQAPLISILNARHTPWALHRMGNPSNPAYMPVIHSRFPVKMNFFQRMINTGWHLYFLYMYFYYGNGEDANKMARKFFGNDMPDINEMVFNTSLLFVNTHFSVDMPYPLVPNCIEIGGIHVKEPQPLPLEIQKFMDEAEHGVIFFTLGSMVRTSTFPNQTIQAFKEAFAELPQRVLWKFENENEDMPSNVLIRKWFPQNDIFGHKNIKAFISHGGNSGALEAVHFGVPIIGIPLFYDQYRNILSFVKEGVAVLLDVNDLTKDNILSSVRTVVNDKSYSERMKALSQLFRDRPMSPLDTAVYWTEYVIRHRGAHHLKTAGAFLHWYQYLLLDVITFLLVTFCAFCFIVKYICKALIHHYWSSSKSEKLKKN

SEQ ID NO: 3 - protein, 467 aa, Streptomyces coelicolor
MPLDAAPVDPASRGPVSAFEPPSSHGADDDDDHRTNASKELFGLKRRVVITGVGVRAPGGNGTRQFWELLTSGRTATRRISFFDPSPYRSQVAAEADFDPVAEGFGPRELDRMDRASQFAVACAREAFAASGLDPDTLDPARVGVSLGSAVAAATSLEREYLLLSDSGRDWEVDAAWLSRHMFDYLVPSVMPAEVAWAVGAEGPVTMVSTGCTSGLDSVGNAVRAIEEGSADVMFAGAADTPITPIVVACFDAIRATTARNDDPEHASRPFDGTRDGFVLAEGAAMFVLEDYDSALARGARIHAEISGYATRCNAYHMTGLKADGREMAETIRVALDESRTDATDIDYINAHGSGTRQNDRHETAAYKRALGEHARRTPVSSIKSMVGHSLGAIGSLEIAACVLALEHGVVPPTANLRTSDPECDLDYVPLEARERKLRSVLTVGSGFGGFQSAMVLRDAETAGAAA

SEQ ID NO: 4 - protein, 407 aa, Streptomyces coelicolor
MSVLITGVGVVAPNGLGLAPYWSAVLDGRHGLGPVTRFDVSRYPATLAGQIDDFHAPDHIPGRLLPQTDPSTRLALTAADWALQDAKADPESLTDYDMGVVTANACGGFDFTHREFRKLWSEGPKSVSVYESFAWFYAVNTGQISIRHGMRGPSSALVAEQAGGLDALGHARRTIRRGTPLVVSGGVDSALDPWGWVSQIASGRISTATDPDRAYLPFDERAAGYVPGEGGAILVLEDSAAAEARGRHDAYGELAGCASTFDPAPGSGRPAGLERAIRLALNDAGTGPEDVDVVFADGAGVPELDAAEARAIGRVFGREGVPVTVPKTTTGRLYSGGGPLDVVTALMSLREGVIAPTAGVTSVPREYGIDLVLGEPRSTAPRTALVLARGRWGFNSAAVLRRFAPTP

SEQ ID NO: 5 - protein, 82 aa, Streptomyces
MTIDDLRRILTECAGEDESVDLGGDILDTPFTELGYDSLALMETAARIEQEFGVAIPDDEFAELATPRAVLAAVSTAVSAAA

SEQ ID NO: 6 - protein, 2051 aa, Saccharomyces cerevisiae
MDAYSTRPLTLSHGSLEHVLLVPTASFFIASQLQEQFNKILPEPTEGFAADDEPTTPAELVGKFLGYVSSLVEPSKVGQFDQVLNLCLTEFENCYLEGNDIHALAAKLLQENDTTLVKTKELIKNYITARIMAKRPFDKKSNSALFRAVGEGNAQLVAIFGGQGNTDDYFEELRDLYQTYHVLVGDLIKFSAETLSELIRTTLDAEKVFTQGLNILEWLENPSNTPDKDYLLSIPISCPLIGVIQLAHYVVTAKLLGFTPGELRSYLKGATGHSQGLVTAVAIAETDSWESFFVSVRKAITVLFFIGVRCYEAYPNTSLPPSILEDSLENNEGVPSPMLSISNLTQEQVQDYVNKTNSHLPAGKQVEISLVNGAKNLVVSGPPQSLYGLNLTLRKAKAPSGLDQSRIPFSERKLKFSNRFLPVASPFHSHLLVPASDLINKDLVKNNVSFNAKDIQIPVYDTFDGSDLRVLSGSISERIVDCIIRLPVKWETTTQFKATHILDFGPGGASGLGVLTHRNKDGTGVRVIVAGTLDINPDDDYGFKQEIFDVTSNGLKKNPNWLEEYHPKLIKNKSGKIFVETKFSKLIGRPPLLVPGMTPCTVSPDFVAATTNAGYTIELAGGGYFSAAGMTAAIDSVVSQIEKGSTFGINLIYVNPFMLQWGIPLIKELRSKGYPIQFLTIGAGVPSLEVASEYIETLGLKYLGLKPGSIDAISQVINIAKAHPNFPIALQWTGGRGGGHHSFEDAHTPMLQMYSKIRRHPNIMLIFGSGFGSADDTYPYLTGEWSTKFDYPPMPFDGFLFGSRVMIAKEVKTSPDAKKCIAACTGVPDDKWEQTYKKPTGGIVTVRSEMGEPIHKIATRGVMLWKEFDETIFNLPKNKLVPTLEAKRDYIISRLNADFQKPWFATVNGQARDLATMTYEEVAKRLVELMFIRSTNSWFDVTWRTFTGDFLRRVEERFTKSKTLSLIQSYSLLDKPDEAIEKVFNAYPAAREQFLNAQDIDHFLSMCQNPMQKPVPFVPVLDRRFEIFFKKDSLWQSEHLEAVVDQDVQRTCILHGPVAAQFTKVIDEPIKSIMDGIHDGHIKKLLHQYYGDDESKIPAVEYFGGESPVDVQSQVDSSSVSEDSAVFKATSSTDEESWFKALAGSEINWRHASFLCSFITQDKMFVSNPIRKVFKPSQGMVVEISNGNTSSKTVVTLSEPVQGELKPTVILKLLKENIIQMEMIENRTMDGKPVSLPLLYNFNPDNGFAPISEVMEDRNQRIKEMYWKLWIDEPFNLDFDPRDVIKGKDFEITAKEVYDFTHAVGNNCEDFVSRPDRTMLAPMDFAIVVGWRAIIKAIFPNTVDGDLLKLVHLSNGYKMIPGAKPLQVGDVVSTTAVIESVVNQPTGKIVDVVGTLSRNGKPVMEVTSSFFYRGNYTDFENTFQKTVEPVYQMHIKTSKDIAVLRSKEWFQLDDEDFDLLNKTLTFETETEVTFKNANIFSSVKCFGPIKVELPTKETVEIGIVDYEAGASHGNPVVDFLKRNGSTLEQKVNLENPIPIAVLDSYTPSTNEPYARVSGDLNPIHVSRHFASYANLPGTITHGMFSSASVRALIENWAADSVSSRVRGYTCQFVDMVLPNTALKTSIQHVGMINGRKLIKFETRNEDDVVVLTGEAEIEQPVTTFVFTGQGSQEQGMGMDLYKTSKAAQDVWNRADNHFKDTYGFSILDIVINNPVNLTIHFGGEKGKRIRENYSAMIFETIVDGKLKTEKIFKEINEHSTSYTFRSEKGLLSATQFTQPALTLMEKAAFEDLKSKGLIPADATFAGHSLGEYAALASLADVMSIESLVEVVFYRGMTMQVAVPRDELGRSNYGMIAINPGRVAASFSQEALQYVVERVGKRTGWLVEIVNYNVENQQYVAAGDLRALDTVTNVLNFIKLQKIDIIELQKSLSLEEVEGHLFEIIDEASKKSAVKPRPLKLERGFACIPLVGISVPFHSTYLMNGVKPFKSFLKKNIIKENVKVARLAGKYIPNLTAKPFQVTKEYFQDVYDLTGSEPIKEIIDNWEKYEQS

SEQ ID NO: 7 - protein, 2073 aa, Schizosaccharomyces pombe
MVEAEQVHQSLRSLVLSYAHFSPSILIPASQYLLAAQLRDEFLSLHPAPSAESVEKEGAELEFEHELHLLAGFLGLIAAKEEETPGQYTQLLRIITLEFERTFLAGNEVHAVVHSLGLNIPAQKDVVRFYYHSCALIGQTTKFHGSALLDESSVKLAAIFGGQGYEDYFDELIELYEVYAPFAAELIQVLSKHLFTLSQNEQASKVYSKGLNVLDWLAGERPERDYLVSAPVSLPLVGLTQLVHFSVTAQILGLNPGELASRFSAASGHSQGIVVAAAVSASTDSASFMENAKVALTTLFWIGVRSQQTFPTTTLPPSVVADSLASSEGNPTPMLAVRDLPIETLNKHIETTNTHLPEDRKVSLSLVNGPRSFVVSGPARSLYGLNLSLRKEKADGQNQSRIPHSKRKLRFINRFLSISVPFHSPYLAPVRSLLEKDLQGLQFSALKVPVYSTDDAGDLRFEQPSKLLLALAVMITEKVVHWEEACGFPDVTHIIDFGPGGISGVGSLTRANKDGQGVRVIVADSFESLDMGAKFEIFDRDAKSIEFAPNWVKLYSPKLVKNKLGRVYVDTRLSRMLGLPPLWVAGMTPTSVPWQFCSAIAKAGFTYELAGGGYFDPKMMREAIHKLSLNIPPGAGICVNVIYINPRTYAWQIPLIRDMVAEGYPIRGVTIAAGIPSLEVANELISTLGVQYLCLKPGSVEAVNAVISIAKANPTFPIVLQWTGGRAGGHHSFEDFHSPILLTYSAIRRCDNIVLIAGSGFGGADDTEPYLTGEWSAAFKLPPMPFDGILFGSRLMVAKEAHTSLAAKEAIVAAKGVDDSEWEKTYDGPTGGIVTVLSELGEPIHKLATRGIMFWKELDDTIFSLPRPKRLPALLAKKQYIIKRLNDDFQKVYFPAHIVEQVSPEKFKFEAVDSVEDMTYAELLYRAIDLMYVTKEKRWIDVTLRTFTGKLMRRIEERFTQDVGKTTLIENFEDLNDPYPVAARFLDAYPEASTQDLNTQDAQFFYSLCSNPFQKPVPFIPAIDDTFEFYFKKDSLWQSEDLAAVVGEDVGRVAILQGPMAAKHSTKVNEPAKELLDGINETHIQHFIKKFYAGDEKKIPIVEYFGGVPPVNVSHKSLESVSVTEEAGSKVYKLPEIGSNSALPSKKLWFELLAGPEYTWFRAIFTTQRVAKGWKLEHNPVRRIFAPRYGQRAVVKGKDNDTVVELYETQSGNYVLAARLSYDGETIVVSMFENRNALKKEVHLDFLFKYEPSAGYSPVSEILDGRNDRIKHFYWALWFGEEPYPENASITDTFTGPEVTVTGNMIEDFCRTVGNHNEAYTKRAIRKRMAPMDFAIVVGWQAITKAIFPKAIDGDLLRLVHLSNSFRMVGSHSLMEGDKVTTSASIIAILNNDSGKTVTVKGTVYRDGKEVIEVISRFLYRGTFTDFENTFEHTQETPMQLTLATPKDVAVLQSKSWFQLLDPSQDLSGSILTFRLNSYVRFKDQKVKSSVETKGIVLSELPSKAIIQVASVDFQSVDCHGNPVIEFLKRNGKPIEQPVEFENGGYSVIQVMDEGYSPVFVTPPTNSPYAEVSGDYNPIHVSPTFAAFVELPGTHGITHGMYTSAAARRFVETYAAQNVPERVKHYEVTFVNMVLPNTELITKLSHTGMINGRKIIKVEVLNQETSEPVLVGTAEVEQPVSAYVFTGQGSQEQGMGMDLYASSPVARKIWDSADKHFLTNYGFSIIDIVKHNPHSITIHFGGSKGKKIRDNYMAMAYEKLMEDGTSKVVPVFETITKDSTSFSFTHPSGLLSATQFTQPALTLMEKSAFEDMRSKGLVQNDCAFAGHSLGEYSALSAMGDVLSIEALVDLVFLRGLTMQNAVHRDELGRSDYGMVAANPSRVSASFTDAALRFIVDHIGQQTNLLLEIVNYNVENQQYVVSGNLLSLSTLGHVLNFLKVQKIDFEKLKETLTIEQLKEQLTDIVEACHAKTLEQQKKTGRIELERGYATIPLKIDVPFHSSFLRGGVRMFREYLVKKIFPHQINVAKLRGKYIPNLTAKPFEISKEYFQNVYDLTGSQRIKKILQNWDEYESS

SEQ ID NO: 8 - protein, 2086 aa, Yarrowia lipolytica
MYPTTGVNTPQSAASLRPLVLSHGQTEHSLLVPTSLYINCTTLRDQFYASLPPATEDKADDDEPSSSTELLAAFLGFTAKTVEEEPGPYDDVLSLVLNEFETRYLRGNDIHAVASSLLQDEDVPTTVGKIKRVIRAYYAARIACNRPIKAHSSALFRAASEDSDNVSLYAIFGGQGNTEDYFEELREIYDIYQGLVGDFIRECGAQLLALSRDHIAAEKIYTKGFDIVKWLEHPETIPDFEYLISAPISVPIIGVIQLAHYAVTCRVLGLNPGQVRDNLKGATGHSQGLITAIAISASDSWDEFYNSASRILKIFFFIGVRVQQAYPSTFLPPSTLEDSVKQGEGKPTPMLSIRDLSLNQVQEFVDATNLHLPEDKQIVVSLINGPRNVVVTGPPQSLYGLCLVLRKQKAETGLDQSRVPHSQRKLKFTHRFLPITSPFHSYLLEKSTDLIINDLESSGVEFVSSELKVPVYDTFDGSVLSQLPKGIVSRLVNLITHLPVKWEKATQFQASHIVDFGPGGASGLGLLTHKNKDGTGVRTILAGVIDQPLEFGFKQELFDRQESSIVFAQNWAKEFSPKLVKISSTNEVYVDTKFSRLTGRAPIMVAGMTPTTVNPKFVAATMNSGYHIELGGGGYFAPGMMTKALEHIEKNTPPGSGITINLIYVNPRLIQWGIPLIQELRQKGFPIEGLTIGAGVPSLEVANEWIQDLGVKHIAFKPGSIEAISSVIRIAKANPDFPIILQWTGGRGGGHHSFEDFHAPILQMYSKIRRCSNIVLIAGSGFGASTDSYPYLTGSWSRDFDYPPMPFDGILVGSRVMVAKEAFTSLGAKQLIVDSPGVEDSEWEKTYDKPTGGVITVLSEMGEPIHKLATRGVLFWHEMDKTVFSLPKKKRLEVLKSKRAYIIKRLNDDFQKTWFAKNAQGQVCDLEDLTYAEVIQRLVDLMYVKKESRWIDVTLRNLAGTFIRRVEERFSTETGASSVLQSFSELDSEPEKVVERVFELFPASTTQIINAQDKDHFLMLCLNPMQKPVPFIPVLDDNFEFFFKKDSLWQCEDLAAVVDEDVGRICILQGPVAVKHSKIVNEPVKEILDSMHEGHIKQLLEDGEYAGNMANIPQVECFGGKPAQNFGDVALDSVMVLDDLNKTVFKIETGTSALPSAADWFSLLAGDKNSWRQVFLSTDTIVQTTKMISNPLHRLLEPIAGLQVEIEHPDEPENTVISAFEPINGKVTKVLELRKGAGDVISLQLIEARGVDRVPVALPLEFKYQPQIGYAPIVEVMTDRNTRIKEFYWKLWFGQDSKFEIDTDITEEIIGDDVTISGKAIADFVHAVGNKGEAFVGRSTSAGTVFAPMDFAIVLGWKAIIKAIFPRAIDADILRLVHLSNGFKMMPGADPLQMGDVVSATAKIDTVKNSATGKTVAVRGLLTRDGKPVMEVVSEFFYRGEFSDFQNTFERREEVPMQLTLKDAKAVAILCSKEWFEYNGDDTKDLEGKTIVFRNSSFIKYKNETVFSSVHTTGKVLMELPSKEVIEIATVNYQAGESHGNPVIDYLERNGTTIEQPVEFEKPIPLSKADDLLSFKAPSSNEPYAGVSGDYNPIHVSRAFASYASLPGTITHGMYSSAAVRSLIEVWAAENNVSRVRAFSCQFQGMVLPNDEIVTRLEHVGMINGRKIIKVTSTNRETEAVVLSGEAEVEQPISTFVFTGQGSQEQGMGMDLYASSEVAKKVWDKADEHFLQNYGFSIIKIVVENPKELDIHFGGPKGKKIRDNYISMMFETIDEKTGNLISEKIFKEIDETTDSFTFKSPTGLLSATQFTQPALTLMEKASFEDMKAKGLVPVDATFAGHSLGEYSALASLGDVMPIESLVDVVFYRGMTMQVAVPRDAQGRSNYGMCAVNPSRISTTFNDAALRFVVDHISEQTKWLLEIVNYNVENSQYVTAGDLRALDTLTNVLNVLKLEKINIDKLLESLPLEKVKEHLSEIVTEVAKKSVAKPQPIELERGFAVIPLKGISVPFHSSYLRNGVKPFQNFLVKKVPKNAVKPANLIGKYIPNLTAKPFEITKEYFEEVYKLTGSEKVKSIINNWESYESKQ

SEQ ID NO: 9 - protein, 317 aa, Escherichia coli
MYTKIIGTGSYLPEQVRTNADLEKMVDTSDEWIVTRTGIRERHIAAPNETVSTMGFEAATRAIEMAGIEKDQIGLIVVATTSATHAFPSAACQIQSMLGIKGCPAFDVAAACAGFTYALSVADQYVKSGAVKYALVVGSDVLARTCDPTDRGTIIIFGDGAGAAVLAASEEPGIISTHLHADGSYGELLTLPNADRVNPENSIHLTMAGNEVFKVAVTELAHIVDETLAANNLDRSQLDWLVPHQANLRIISATAKKLGMSMDNVVVTLDRHGNTSAASVPCALDEAVRDGRIKPGQLVLLEAFGGGFTWGSALVRF

SEQ ID NO: 10 - protein, 403 aa, Aloe arborescens
MSSLSNASHLMEDVQGIRKAQRADGTATVMAIGTAHPPHIFPQDTYADFYFRATNSEHKVELKKKFDRICKKTMIGKRYFNYDEEFLKKYPNITSFDEPSLNDRQDICVPGVPALGAEAAVKAIAEWGRPKSEITHLVFCTSCGVDMPSADFQCAKLLGLRTNVNKYCVYMQGCYAGGTVMRYAKDLAENNRGARVLVVCAELTIIGLRGPNESHLDNAIGNSLFGDGAAALIVGSDPIIGVEKPMFEIVCAKQTVIPNSEDVIHLHMREAGLMFYMSKDSPETISNNVEACLVDVFKSVGMTPPEDWNSLFWIPHPGGRAILDQVEAKLKLRPEKFRATRTVLWDCGNMVSACVLYILDEMRRKSADEGLETYGEGLEWGVLLGFGPGMTVETILLHSLPLM

SEQ ID NO: 11 - protein, 393 aa, Hypericum perforatum
MGSLDNGSARINNQKSNGLASILAIGTALPPICIKQDDYPDYYFRVTKSDHKTQLKEKFRRICEKSGVTKRYTVLTEDMIKENENIITYKAPSLDARQAILHKETPKLAIEAALKTIQEWGQPVSKITHLFFCSSSGGCYLPSSDFQIAKALGLEPTVQRSMVFPHGCYAASSGLRLAKDIAENNKDARVLVVCCELMVSSFHAPSEDAIGMLIGHAIFGDGAACAIVGADPGPTERPIFELVKGGQVIVPDTEDCLGGWVMEMGWIYDLNKRLPQALADNILGALDDTLRLTGKRDDLNGLFYVLHPGGRAIIDLLEEKLELTKDKLESSRRVLSNYGNMWGPALVFTLDEMRRKSKEDNATTTGGGSELGLMMAFGPGLTTEIMVLRSVPL

SEQ ID NO: 12 - protein, 169 aa, Streptomyces
MRHVEHTVTVAAPADLVWEVLADVLGYADIFPPTEKVEILEEGQGYQVVRLHVDVAGEINTWTSRRDLDPARRVIAYRQLETAPIVGHMSGEWRAFTLDAERTQLVLTHDFVTRAAGDDGLVAGKLTPDEAREMLEAVVERNSVADLNAVLGEAERRVRAAGGVGTVTA

SEQ ID NO: 13 - protein, 256 aa, Streptomyces
MSGRKTFLDLSFATRDTPSEATPVVVDLLDHVTGATVLGLSPEDFPDGMAISNETVTLTTHTGTHMDAPLHYGPLSGGVPAKSIDQVPLEWCYGPGVRLDVRHVPAGDGITVDHLNAALDAAEHDLAPGDIVMLWTGADALWGTREYLSTFPGLTGKGTQFLVEAGVKVIGIDAWGLDRPMAAMIEEYRRTGDKGALWPAHVYGRTREYLQLEKLNNLGALPGATGYDISCFPVAVAGTGAGWTRVVAVFEQEEED